(19) United States
(12) Patent Application Publication
(10) Pub. No.: US 2007/0212314 A1
(43) Pub. Date: Sep. 13, 2007

(54) Silicone Adhesive Formulation Containing An Antiperspirant

(75) Inventors: Kevin Patrick Murphy, Midland, MI (US); Gerald Kenneth II Schalau, Freeland, MI (US)

Correspondence Address: DOW CORNING CORPORATION CO1232, 2200 W. SALZBURG ROAD, P.O. BOX 994, MIDLAND, MI, 48686-0994, US

(73) Assignee: DOW CORNING CORPORATION

(21) Appl. No.: 11/658,133

(22) PCT Filed: Jul. 27, 2005
(86) PCT No.: PCT/US05/27126

§ 371 Date: Jan. 22, 2007

Related U.S. Application Data
(60) Provisional application No. 60/607,667, filed on Sep. 7, 2004.

(51) Int. Cl. A61K 8/25

(52) U.S. Cl. 424/66; 106/287.1; 424/400; 424/65; 424/68

(57) ABSTRACT

This invention relates to a method of enhancing the adhesion of a silicone containing adhesive formulation. The method comprises mixing 1 to 99.9 wt. % of a silicone-containing adhesive formulation and 0.1 to 50 wt. % antiperspirant compound to form a composition. The composition is then applied onto a surface and allowed to cure. The invention also relates to a composition comprising 1 to 99.9 wt. % of a silicone-containing adhesive formulation and 0.1 to 50 wt. % antiperspirant salt. The resultant adhesive films are especially useful in healthcare and cosmetic applications where they inhibit perspiration and, thus, retain the adhesive strength of the adhesive.

### BACKGROUND OF THE INVENTION

The present invention relates to silicone adhesive formulations that contain an antiperspirant. These formulations can be used in conventional antiperspirant applications or they can be used in adhesive applications where moisture inhibits or decreases the adhesion of the adhesive formulation.

The use of antiperspirants is well known in the personal care market. Typically, the antiperspirants comprise salts of aluminum and/or zirconium. It is also known in the art to incorporate silicones into formulations containing such salts to improve the aesthetics of such formulations and aid in the delivery of the salts.

Silicone adhesives are also well known in the art. They are useful on a variety of substrates such as paper, plastic, rubber, and the like. Such adhesives are also known to be useful for adherence to the human or animal body in applications such as transdermal drug delivery devices, dressings, prosthesis and the like.

One problem with adhesives is that adhesive strength can be reduced in the presence of moisture. For example, the adhesion and the wear time of a bandage or medical device attached with a silicone adhesive can be reduced when the patient perspires. This can result in failure of the adhesive and, thus, delamination from the skin. In the case of a wound dressing, this can result in slower wound healing and further potential for infection. Additionally, it can result in increased cost due to the patient or caregiver having to replace the dressing more frequently.

One traditional approach to wet skin adhesion has been to utilize an adhesive that has a higher adhesive strength so that even when wet, it will maintain sufficient adherence. Such high adhesive strength, however, often results in skin damage when the adhesive is removed. This is especially a problem when the adhesive needs to be applied to compromised or damaged skin or when the adhesive device needs to be applied and reapplied several times to the same area.

It is also known that when an adhesive is placed over compromised skin such as wounds, periwound skin, and scars, excess moisture in or around the wound bed from sources such as perspiration is known to cause maceration of the skin. Traditional methods of eliminating this excess moisture have been reactive rather than proactive and have focused on methods of increasing the moisture vapor transmission rate (MVTR) of the dressing, drape or tape. Steps taken to increase the MVTR include treatments of the substrate or primary component of the dressing construct including the use of breathable substrates and perforated or absorbant substrates. Other alternative steps to increase MVTR have included adjusting the adhesive coverage via pattern coating, or lesser adhesive coat weights.

We have now discovered that by adding an antiperspirant to a silicone adhesive, the adhesion of the adhesive can be improved by reducing the amount of moisture present at the site of the adhesive. Therefore, less aggressive adhesives can be used and, thus, skin trauma is limited when the adhesive is removed. Moreover, use of the composition of this invention also solves the problem of perspiration in a wound bed by reducing the amount of moisture generated beneath an adhesive and, thereby, reducing the necessity of high dressing MVTR and reducing the chance for infections that may be facilitated by excessive moisture. Finally, antiperspirant salts are known to have antibacterial effects and, thus, can prevent bacterial contamination in the area of the adhesive.

### SUMMARY OF THE INVENTION

This invention relates to a method of enhancing the adhesion of a silicone containing adhesive formulation. The method comprises mixing 1 to 99.9 wt. % of a silicone-containing adhesive formulation and 0.1 to 50 wt. % antiperspirant compounds. The composition is then applied onto a surface and allowed to cure. The invention also relates to a composition comprising 1 to 99.9 wt. % of a silicone-containing adhesive formulation and 0.1 to 50 wt. % antiperspirant compounds. The resultant films are especially useful as adhesives in healthcare and personal care applications, especially on the skin, where they can serve, for example, as, on or in topical drug delivery systems, masking systems for skin protection in dermal treatments, wound dressings and bandages for minor wounds, burns, acute and chronic wounds, skin sealants, skin protective films, scar treatments, exfoliation and hair remover products, deodorizing films, antiperspirant active and fragrance delivery systems, anti-wrinkle patches, moisturizing masks and the like. These adhesives have benefits in topical therapies, wound care, surgical closure, scar care, underarm care, foot care, body and skin care, cosmetics, make-up, foundations and the like. They can likewise be used on other biological surfaces such as hair, nails, teeth, eyes, and mucous membranes as well as similar applications on animals other than humans.

### DETAILED DESCRIPTION OF THE INVENTION

The present invention relates to adhesive compositions that do not degrade in adhesive strength in the presence of moisture. These compositions comprise an adhesive formulation and an antiperspirant compound.

As used in the present invention:
A “silicone-containing adhesive formulation” means a formulation which bonds to a substrate or which is capable of bonding substances together by surface attachment in which silicone is an agent in the formulation.
An “antiperspirant” is a substance having an astringent action that tends to reduce the size of skin pores and thus restrain the passage of moisture on local body areas.

The adhesive formulation used in this composition is not critical. It can comprise any silicone-containing adhesive formulation including, for example, elastomers that cure by hydrosilylation or condensation, silicone-containing pressure sensitive adhesives such as those based on silanol containing polydiorganosiloxanes and silanol containing silicone resins, and silicone-containing hot melt adhesives.

One reperesentative silicone-containing adhesive formulation comprises at least one polydiorganosiloxane having silicon-bonded alkenyl groups, at least one hydrosilicon compound having silicon bonded hydrogen atoms and a catalyst for the reaction of the Si—H groups with the Si-alkenyl groups. This type of material is described, for example, in U.S. Pat. Nos. 4,991,574 and 3,020,260, which are incorporated herein by reference. The material of this reference can comprise (A) a polydiorganosiloxane having on average two silicon-bonded alkenyl groups per molecule, said alkenyl group having from 2 to 6 carbon atoms and no silicon atom having more than one alkenyl group bonded thereto, the remaining silicon-bonded organic groups being selected from alkyl and aryl groups, said polydiorganosiloxane having a viscosity at 25° C. of from 50 to 10,000 mm²/s, (B) a hydrosilicon compound having at least 3 silicon-bonded hydrogen atoms per molecule and consisting essentially of RHSiO— groups, R₂XSiO_{1/2} groups and optionally R₂SiO groups and having a viscosity at 25° C. of no more than 1000 mm²/s, wherein R denotes an alkyl or aryl group having no more than 8 carbon atoms, and X denotes H or R, (C) a diorganohydrogensiloxy-terminated polydiorganosiloxane, having a viscosity at 25° C. of from 1 to 200 mm²/s, wherein the organic substituents are alkyl or aryl groups having no more than 8 carbon atoms and (D) a catalyst for the reaction of the SiH groups with Si-alkenyl groups.

Polydiorganosiloxane (A) for use in the above adhesive formulation can have on average two silicon-bonded alkenyl groups per molecule, each alkenyl group being bonded to a different silicon atom. The polydiorganosiloxane (A) can be a substantially linear polymer, although a small amount of branching may be present. The alkenyl groups can be attached to silicon atoms that are distant from each other in the molecule, and alternatively they are attached to the terminal silicon atoms of the siloxane chain. The alkenyl groups can have a maximum of 6 carbon atoms and may be for example vinyl, allyl or hexenyl groups. The remaining organic substituents of polydiorganosiloxane (A) are selected from alkyl and aryl groups, alternatively alkyl groups having no more than 8 carbon atoms or phenyl groups. Examples of such remaining substituents are methyl, ethyl, propyl, isobutyl and phenyl. In one embodiment the polydiorganosiloxane compounds have at least 50%, alternatively substantially all, of the remaining organic substituents are methyl groups. Polydiorganosiloxane (A) can have a viscosity at 25° C. of from 50 to 10,000 mm²/s. In one embodiment, the viscosity range for polydiorganosiloxane (A) is from 100 to 1000 mm²/s at 25° C. A polydiorganosiloxane with a viscosity above 10,000 mm²/s would be difficult to use at room temperature where it needs to be mixed with the other components for the reaction. In one embodiment, the viscosity is chosen such that the composition is readily flowable prior to curing and can be dispensed easily from a container. Polydiorganosiloxanes (A) are known in the art and many are commercially available. They may be prepared for example by equilibrating in the presence of a mild catalyst, e.g. toluene sulphonic acid or acid treated clay, cyclic siloxanes with low molecular weight vinyl substituted end-blockers, which are produced by hydrolysing the appropriate chlorosilanes, e.g. vinyldiorganochlorosilane with diorganodichlorosilanes.

The hydrosilicon compound (B) for use in the above adhesive formulation can be an organosiloxane, having at least 3 silicon-bonded hydrogen atoms per molecule. These hydrogen atoms may be located at terminal siloxane units as well as at siloxane units in the polymer chain, or they may be located only within the siloxane chain. This hydrosilicon compound is essentially composed of units of the general formula RHSiO— groups, R₂XSiO_{1/2} groups and optionally R₂SiO groups, wherein R and X are as defined above. The hydro-silicon compound (B) can be a linear siloxane polymer, which may consist of units of the formula RHSiO— and R₃SiO_{1/2} and/or R₂HSiO_{1/2} or it may consist of R₂SiO—, RHSiO— and R₃SiO_{1/2} and/or R₂HSiO_{1/2} units. In one embodiment, hydrosilicon compounds wherein not more than 50% of the units have silicon-bonded hydrogen atoms are used. The viscosity of the hydrosilicon compound (B) can be those that do not exceed 1000 mm²/s at 25° C., alternatively viscosities below 500 mm²/s may be used to facilitate mixing with the other components of the composition. In one embodiment, the viscosity is less than 50 mm²/s at 25° C. The hydrosilicon compounds (B) are well known in the art, and may be produced according to known methods. One such method consists of equilibrating a hydrogen cyclopolysiloxane with a cyclic organopolysiloxane, e.g. polydimethylcyclosiloxane and triorganosiloxy end-blocking compounds.

The diorganohydrogensiloxy-terminated polydiorganosiloxane (C) of the above adhesive formulation can comprise diorganosiloxy units and diorganohydrogen siloxy units. The organic substituents are alkyl or aryl groups having no more than 8 carbon atoms, alternatively methyl or phenyl. Compound (C) can have a viscosity at 25° C. of from 1 to 200 mm²/s, alternatively from 5 to 50 mm²/s. This compound will react in the gel forming composition via its endgroups only.

Component (D) of the above adhesive formulation is a catalyst for the reaction of SiH groups with Si-alkenyl groups. Such catalysts are generally group VIII metals or complexes or compounds thereof. In one embodiment, component (D) is a platinum compound or complex. This component is effective in catalysing the addition reaction between the alkenyl groups in (A) and the silicon-bonded hydrogen atoms in (B) and (C). The addition reaction between SiH groups and unsaturated aliphatic groups is well known in the art of organosilicon chemistry as are a variety of platinum-based catalysts for the reaction. Such catalysts are well documented in the art and include chloroplatinic acid, platinum acetylacetonate, complexes of platinous halides with unsaturated compounds such as ethylene, propylene, organovinylsiloxanes and styrene, hexamethyldiplatinum, PtCl₂; PtCl₃; and Pt(CN₃). In one embodiment, the catalysts are complexes of platinum compounds and vinyl siloxanes e.g. those formed by the reaction of chloroplatinic acid and divinyltetramethyl disiloxane. A sufficient amount of the catalyst should be employed to provide a homogenous and effective cure of the composition. The proportion of platinum catalyst is usually that which will provide from 1 to 200 parts by weight of Pt per million parts of the combined weights of (A), (B) and (C).

The value of RHAlk, i.e. the ratio of the number of silicon-bonded hydrogen atoms in (B) and (C) with respect to the number of silicon bonded alkenyl groups in (A), can be such that the desired gel is formed. For example, there can be excess alkenyl groups (e.g., between 1:1.1 and 1:20 or between 1:1 and 1:3) or there can be excess SiH (e.g., between 1.1:1 and 20:1 or between 3:1 and 1:1) or an approximately equal number (i.e., about 1:1). It should be noted that other silicone-containing adhesive formulation comprising a polydiorganosiloxane having silicon-bonded alkenyl groups, a hydrosilicon compound having silicon bonded hydrogen atoms and a catalyst for the reaction of the Si—H groups with the Si-alkenyl groups can be used in this invention.

The adhesive compositions of the invention can be prepared by simply mixing the individual components (A) to (D) in any order. Generally the compositions will cure to the desired adhesive product at temperatures of about 25° C. If desired, however, curing may be accelerated by exposure to elevated temperatures, e.g. above 150° C. In order to maintain the compositions in the uncured state prior to use, for example during storage or transportation, they may be packaged in two or more parts. A first part may comprise at least part of Component (A) together with Component (D), while a second part may comprise Components (B) and (C) together with any remainder of component (A). These packages can take any suitable form, for example bottles, sachets or pressurized packs, e.g. aerosol cans. The contents of the packages are mixed together in predetermined ratios prior to their use, for example by manual mixing or by being dispensed via a mixing valve from a can-in-can or a can-upon-can system. Such systems are known in the art and are described e.g. in G. B. Patent Specification 2 185 750.

If desired, the above adhesive composition can also contain at least one hydroxy-substituted siloxane resin. This resin increases the adhesion of the adhesive to a medical substrate or the skin. This resin comprises R₃SiO_{1/2} units (M units) and SiO_{4/2} units (Q units) wherein each R is independently a linear, branched or cyclic hydrocarbon group having 1-20 carbon atoms. R can be unsubstituted or substituted with halogen atoms. Each R can be identical or different, as desired. The hydrocarbon group of R can be exemplified by alkyl groups such as methyl, ethyl, propyl, butyl, hexyl, octyl3,3,3-trifluoropropyl, chloromethyl, and decyl, alkenyl groups such as vinyl and hexenyl, cycloaliphatic groups such as cyclohexyl, aryl groups such as phenyl, tolyl, and xylyl, chlorophenyl, and aralkyl groups such as benzyl, styryl and alpha-methylstyryl. Alternatively, each R group is an independently selected alkyl or alkenyl group comprising 1 to 8 carbon atoms or aryl group comprising 6 to 9 carbon atoms. Alternatively, each R group is independently selected from methyl and vinyl.

If an alkenyl group is present in the above resin, typically the mole % of R groups present as alkenyl groups is less than 10%, alternatively less than 5%. For example, if the resin contains vinyl groups, typically they are present in an amount of less than 5 wt. % of the resin solids, alternatively less than 2.5 wt. % of the resin solids, alternatively 1.5-2 wt. % of the resin solids.

The molar ratio of R₃SiO_{1/2} (M units) to SiO_{4/2} (Q units) in the above resin can be from 0.6:1 to 4:1. Alternatively, the molar ratio of M:Q can be from 0.6:1 to 1.9:1. Alternatively, the molar ratio of M:Q can be from 0.6:1 to 1.0:1. The resins can also contain triorganosiloxy units (T units), for example 0.5 to 1 triorganosiloxy group for every SiO_{4/2} unit, alternatively 0.6 to 0.9 triorganosiloxy group for every SiO_{4/2} unit.

It should be noted that more than 1 of the above resins could be included in the present invention. In this case, at least one of the resins should have the silanol content as described below but, by the same token, one could have the silanol capped so that there is substantially no silanol present.

In one embodiment a majority of all R groups in the above resin are methyl and the total number of R groups that have olefinic unsaturation is no more than 0.5% of all R groups. In another embodiment substantially all of the R groups are methyl. In another embodiment substantially all of the R groups are substantially free of olefinic unsatuaration. In yet another embodiment of the invention, 2 resins are included—one in which substantially all of the R groups are methyl and the other in which 3.5 to 4 mole % of the R groups are vinyl and substantially all of the remaining R groups are methyl.

The above resins also contain silicon-bonded hydroxyl groups ranging from about 0.01 up to 5 weight percent of the resin, alternatively from about 1 to about 5 wt. % of the resin.

The above resins comprising R₃SiO_{1/2} units and SiO_{4/2} units are well known in the art. These copolymers are described, for example, in U.S. Pat. Nos. 3,936,582, 2,676,182, and 2,857,356. The resinous copolymers can be prepared by cohydrolysis of a mixture of silanes having four hydrolyzable groups, e.g., silicon tetrachloride, and triorganosilanes having one hydrolyzable group, e.g., trimethylchlorosilane, in the proper ratio. A specific method for the preparation of these resinous copolymers is described in U.S. Pat. No. 2,676,182, wherein a silica hydrosol is reacted under acidic conditions with a source of triorganosiloxy units such as a hexaorganodisiloxane, for example, hexamethyldisiloxane, or a hydrolyzable triorganosilane, for example, trimethylchlorosilane, or mixtures thereof.

The above resins can be used in the adhesive of this invention in an amount of 2-45 wt. % based on the weight of the composition, alternatively 5-40 wt. % and alternatively 10-35 wt. %.

Another adhesive formulation useful in the present invention is that made by the condensation reaction of a polydiorganosiloxane having silanol terminal functionality and a silanol containing silicone resin as described, for example, in U.S. Pat. Nos. 4,591,622, 4,584,355, 4,655,767 and RE35474, all of which are hereby incorporated by reference.

The organic substituents of the silanol-terminated polydiorganosiloxane are generally alkyl groups having 1 to 6 carbon atoms or phenyl groups. In one embodiment, at least 80 percent of the organic substituents are methyl groups. In another embodiment, the silanol-terminated polydiorganosiloxane is a dimethylhydroxy-terminated polydimethylsiloxane. The silanol-terminated polydiorganosiloxane can have a viscosity of at least 0.1 Pa·s and can have viscosity up to 30000 Pa·s or higher. It can for example be prepared by the method of U.S. Pat. No. 5,319,120, U.S. Pat. Nos. 2,490,357, 2,542,334, 2,927,907, 3,002,951, 3,161,614, 3,186,967, 3,509,191, and 3,697,473, all of which are incorporated herein by reference.

The silanol-containing silicone resin is generally a non-linear siloxane resin and can consist of siloxane units of the formula R′ₐSiO_{4-a/2} wherein R′ denotes a hydroxyl, hydrocarbon or hydrocarbonoxy group and wherein has an average value of from 1 to 1.8. The resin can comprise monovalent trihydrocarbonsiloxy (M) groups of the formula R″₃SiO_{1/2} and tetrafunctional (Q) groups SiO_{4/2}, wherein R″ denotes a monovalent hydrocarbon group having 1 to 6 carbon atoms, alternatively a methyl group. The number ratio of M groups to Q groups can be in the range 0.5:1 to 1.2:1 (equivalent to a value of a in the formula R′ₐSiO_{4-a/2} of 1.0 to 1.63), and alternatively is 0.6:1 to 0.9:1. The silicone resin can contain at least 0.2 percent by weight up to about 3 or 5 percent silicon-bonded hydroxy radicals. These can be present as dimethylhydroxysiloxy (HO)(CH₃)₂SiO_{1/2} units. The resin may be prepared according to Daudt et al., U.S. Pat. No. 2,676,182 which is hereby incorporated by reference.

The silicone pressure sensitive adhesive can comprise 20 to 80 parts by weight, alternatively 30 to 60 parts, of the silanol-terminated polydiorganosiloxane of Tg below −20° C., and 80 to 20 parts by weight, alternatively 70 to 40 parts, of the silanol-containing silicone resin of Tg above 0° C. Alternatively, the silicone pressure sensitive adhesive is the product of mixing 30 to 60 parts by weight of a silanol-terminated polydiorganosiloxane of Tg below −20° C. and viscosity 0.1-30000 Pa·s at 25° C. with 40 to 70 parts by weight of a silanol-containing silicone resin of Tg above 0° C. comprising monovalent trihydrocarbonsiloxy (M) groups of the formula R′₃ SiO_{1/2} and tetrafunctional (Q) groups SiO_{4/2} wherein R″ denotes a monovalent hydrocarbon group having 1 to 6 carbon atoms, the number ratio of M groups to Q groups being in the range 0.5:1 to 1.2:1.

When mixed, the silanol groups of the polydiorganosiloxane generally undergo some condensation reaction with the silanol groups of the silicone resin so that the polydiorganosiloxane is crosslinked by the silicone resin (that is, polydiorganosiloxane chains are bonded together through resin molecules to give chain branching and entanglement and/or a small amount of network character) to form the silicone pressure sensitive adhesive. A catalyst, for example an alkaline material such as ammonia, ammonium hydroxide or ammonium carbonate can be mixed with the silanol-terminated polydiorganosiloxane and the silicone resin to promote this crosslinking reaction. It may for example be preferred, particularly for personal care and medical applications, that the silanol-terminated polydiorganosiloxane have viscosities in the range 1-100 Pa·s at 25° C. and are crosslinked with the aid of a catalyst. The amounts of silanol-terminated polydiorganosiloxane and silanol-containing silicone resin are often such that the Tg of the product of mixing is between −15 and 15° C. (T at tan delta maximum). The silanol-containing silicone resin often lowers the rubbery plateau modulus (G′ at Tg+30° C.) of the blend below 7E+05 dyne/cm2; this is an indication that the network character is crosslinked.

The silicone pressure sensitive adhesive produced by mixing the silanol-terminated polydiorganosiloxane and silanol-containing silicone resin may be chemically treated to react silanol groups with an endblocking agent which introduces triorganosilyl units, as described in U.S. Pat. No. 4,655,767 which is hereby incorporated by reference. The endblocking agent can, for example, be a disilazane such as hexamethyldisilazane or a trialkyl alkoxy silane such as trimethyl ethoxy silane or trimethyl methoxy silane. Reaction with such an end blocking agent reduces the sensitivity of the adhesive to loss of adhesion in contact with reagents such as amines.

Another adhesive formulation useful in the present invention comprises a hot-melt silicone pressure sensitive adhesive. Such compositions are known in the art such as, for example, U.S. Pat. Nos. 5,330,747 and 5,162,410 which are hereby incorporated by reference. Typically, such compositions comprise a mixture of a trimethylsilyl-endblocked resinous copolymer containing silicon bonded hydroxyl radicals a silanol-endblocked polydiorganosiloxane fluid, and a polysiloxane fluid.

One specific example of a hot melt silicone adhesive formulation comprises: (i) 40 to 70 parts by weight of a trimethylsilyl-endblocked low molecular weight hydrocarbon or benzene-soluble resinous copolymer containing silicon bonded hydroxyl radicals and consisting essentially of triorganosiloxy units of the formula R″″₃ SiO_{1/2} and tetrafunctional siloxy units of the formula SiO_{4/2} in a ratio of 0.6 to 0.9 triorganosiloxy units per tetrafunctional siloxy unit present in the copolymer molecule, and R″″ is a monovalent hydrocarbon radical of one to six carbon atoms; (ii) 30 to 60 parts by weight of a silanol-endblocked polydiorganosiloxane fluid, wherein the total parts by weight of the resinous copolymer and the silanol-endblocked polydiorganosiloxane fluid equals 100 parts; the mixture of (i) and (ii) exhibiting tackiness and adhesiveness, and blended with: (iii) 0.5 to 20 weight percent, based on the total weight of the resinous copolymer and the silanol-endblocked polydiorganosiloxane fluid, of a phenyl-containing polysiloxane fluid of the formula A₃SiO[Si(C₆H₅)(R′)O]ₓ [Si(R₂O]_{y} SiB₃ wherein R is a monovalent radical selected from the group consisting of —OSiR″₃, hydrocarbon radicals of one to three carbon atoms, and —OH; R′ is a monovalent radical selected from the group consisting of —OSiR″₃, —OH and —CH₃; R″ is a monovalent hydrocarbon radical of one to three carbon atoms; A and B are endblocking units selected from the group consisting of —OSiR′″₃ where R′″ is a hydrocarbon radical of one to eight carbon atoms, —OH, halide radicals, and amine radicals; x is an integer having a value greater than zero; and the value of x and y is such that the phenyl-containing polysiloxane fluid has a viscosity at twenty-five degrees Centigrade of 5 to 60,000 centistokes, and has 1 to 100 phenyl groups per 100 siloxane units.

Details of the adhesives and examples of a process for its manufacture are set forth in U.S. Pat. No. 5,162,410 noted previously, and reference may be had thereto. The adhesive is available commercially from Dow Corning Corporation, Midland, Mich., USA.

The adhesive formulations of the present invention may also include diluents. Such diluents are often necessary to decrease the viscosity of the formulation sufficiently for application. Examples of diluents include silicon containing diluents such as hexamethyldisiloxane, octamethyltrisiloxane, and other short chain linear siloxanes, cyclic siloxanes such as octamethylcyclotetrasiloxane and decamethylcyclopentasiloxane, dodecamethylcyclohexasiloxane, organic diluents such as butyl acetate, alkanes, alcohols, ketones, esters, hydrofluorocarbons or any other material which can dilute the formulation without adversely affecting any of the component materials of the formulation or the curing time. The above diluents can be used in amounts of up 95 wt. % of the formulation. On application, however, the diluent often substantially volatilizes leaving the other component materials on the desired site.

The composition of the invention also includes antiperspirant compounds. The antiperspirant compound of the present invention can comprise an antiperspirant suitable for application to human skin. The active in the composition may be solubilized or in the form of solid particulates or dispersed liquid droplets. The concentration of antiperspirant in the final composition should be sufficient to provide the desired moisture control.

The final composition of the invention can comprise antiperspirant at concentrations ranging from about 0.1% to about 50%, alternatively from about 5% to about 35%, alternatively from about 7% to about 30%, by weight of the composition. These weight percentages are calculated on an anhydrous metal salt basis exclusive of water and any complexing agents such as glycine, glycine salts, or other complexing agents.

The antiperspirant compound for use in the compositions of the present invention includes any compound, composition or other material having antiperspirant activity. Antiperspirant compounds include astringent metallic salts, especially the inorganic and organic salts of aluminum zirconium and zinc, as well as mixtures thereof. These include aluminum containing and/or zirconium-containing materials or salts, such as aluminum halides, aluminum chlorohydrate, aluminum hydroxyhalides, zirconyl oxyhalides, zirconyl hydroxyhalides, and mixtures thereof. Examples include aluminum-zirconium tetrachlorohydrex and aluminum-zirconium trichlorohydrex.

The aluminum salts for use in compositions of the invention include those that conform to the formula
Al₂(OH)ₐCl_{b}.xH₂O
wherein a is from about 2 to about 5; the sum of a and b is about 6; x is from about 1 to about 6; and wherein a, b, and x may have non-integer values. In one embodiment the aluminum chlorohydroxides are those wherein a=5, and wherein a=4. Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980, all of which are incorporated herein by reference. Mixtures of aluminum salts are described in British Patent Specification 1,347,950, Shin et al., published Feb. 27, 1974, which description is also incorporated herein by reference.

Zirconium salts for use in the compositions of the invention include those which conform to the formula:
ZrO(OH)₂₋ₐClₐ.xH₂O
wherein a is from about 1.1 to about 2.0; x is from about 1 to about 8; and wherein a and x may both have non-integer values. These zirconium salts are described in Belgian Patent 825,146, to Schmitz, issued Aug. 4, 1975, which description is incorporated herein by reference. Suitable zirconium salts are those complexes which additionally contain aluminum and glycine, commonly known as ZAG complexes. These ZAG complexes contain aluminum chlorohydroxide and zirconyl hydroxy chloride conforming to the above described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978, all of which are incorporated herein by reference.

The present composition can also comprise hygroscopic fillers such as silica, clay, alginate, collagen, chitosan, and hydrophilic organic fillers such as acrylic polymers polyacrylic acid, polyvinyl alcohol and polyvinylpyrolidone, and polyethylene glycol as well as larger polysaccharides and materials of cellulose origin to increase the moisture absorptive capability of the adhesive. In addition, other fillers can be used if desired. These include, for example, ground, precipitated, and colloidal calcium carbonates which can be untreated or treated with stearate or stearic acid; reinforcing silicas such as fumed silicas, precipitated silicas, and hydrophobed silicas; crushed quartz, ground quartz, alumina, aluminum hydroxide, titanium dioxide, diatomaceous earth, iron oxide, carbon black, and graphite. One class of fillers are synthetic silicas where the surfaces of the silica are modified with silicon compounds to produce a hydrophobic behavior. These materials differ from one another in surface area, the silicon compound used to treat the silica, and the extent of surface treatment. Such materials are surprisingly able to reduce the viscosity of the film forming formulation. In addition, resinous reinforcing fillers can be used herein to form transparent films. Silica, calcium carbonate, andresinous fillers can be used in one embodiment of the invention. Specific examples include Cab-O-Sil@ TS-530 treated filler, Aerosil@ R8200 treated filler, and Wacker HDX H2000 treated filler.

The present composition can also comprise a variety of active agents. The active agents used in the present invention are generally not critical. They can comprise any solid or liquid material that can be formulated in the composition or coated on the composition by, for example, plasma, and subsequently released at the desired rate. The active agent should also not interfere with the curing of the silicone formulation to an unacceptable extent. Suitable active agents include cosmetics, personal care, cosmeceuticals, therapeutic or diagnostic materials, fragrances, natural extracts, aloe vera, onion, collagen, pesticides, herbicides, and the like. These can include, for example, anti-microbial agents including silver, copper and bactericidal and/or fungicidal chemicals excluding those of biological origin.

Therapeutic active agents which may be employed include, for example, anti-acne agent, antibiotic, antiseptic, anti-fungal, antibacterial, antimicrobial, biocides, anti-inflammatory, astringents, hormones, anti-cancer agents, smoking cessation compositions, cardiovascular, histamine blocker, bronchodilator, analgesic, anti-arrythmic, antihistamine, alpha-I blocker, beta blocker, ACE inhibitor, diuretic, anti-aggregant, sedative, tranquillizer, anti-convulsant, anti-coagulant agents, vitamins, anti-aging agents, agents for treating gastric and duodenal ulcers, anti-cellulites, proteolytic enzymes, healing factors, cell growth nutrients, peptides and others. Specific examples of suitable therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol.

In addition to the therapeutic or diagnostic materials, active, agents could be cosmetics such as perfumes, UV protectors, shaving products, deodorants or the like. Suitable cosmetics are known to those skilled in the art.

The proportion of the active agent employed in the present invention is chosen in accordance with the concentration of the active agent required in the composition to deliver the dosage required at the proposed delivery rate. This may vary within a wide range such as from 0.1 to about 70 weight percent, alternatively 0.1 to 20 weight percent, of the final composition.

If desired the formulation may also contain other additional ingredients. These include colorants, colored indicators, other diluents, extenders such as silicone fluids, silicone resins, excipients employed in pharmacy, compounds intended to perform as pH buffers in controlling the environment immediately in and around the formulation, stabilizers, preservatives, surfactants for cellular formulations such as fluorinated silicones, processing aids such as cyclic or linear polydiorganosiloxanes, bioadhesive materials, and hydrophilic, modulating and swellable components or polymers as set forth in EP Publication 465,744.

Some additional examples of the cosmetics, personal care, and cosmeceutical ingredients and pharmaceutical excipients that may be used herein may be found in the CTFA ingredient Database and the handbook of pharmaceutical excipients and can include, for example, absorbents, anti-cacking agents, antioxidants, antistatic agents, astringents, binders, buffering agents, bulking agents, chelating agents, colorants, cosmetic astringents, cosmetic biocides, deodorant agents, emollients, external analgesics, film formers, flavoring agents, fragrance ingredients, humectants, lytic agents, moisturizing agents, occlusivity enhancers, opacifying agents, oxidizing and reducing agents, penetration enhancers, pesticides, plasticizers, preservatives, skin bleaching agents, skin conditioning agents, skin protectants, slip modifiers, solubilizing agents, solvents, sunscreen agents, surface modifiers, surfactants and emulsifying agents, suspending agents, thickening agents, viscosity controlling agents including increasing or decreasing agents, UV light absorbers,

Cosmetic, personal care and cosmeceutical ingredients, and pharmaceutical excipients which may be employed are selected, for example, from the following chemical classes: alcohols, fatty alcohols and polyols, aldehydes, alkanolamines, alkoxylated alcohols (e.g. polyethylene glygol derivatives of alcohols and fatty alcohols), alkoxylated amides, alkoxylated amines, alkoxylated carboxylic acids, amides including salts (e.g. ceramides), amines, amino acids including salts and alkyl substituted derivatives, esters, alkyl substituted and acyl derivatives, polyacrylic acids, acrylamide copolymers, adipic acid copolymers, alcohols, aminosilicones, biological polymers and derivatives, butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, TiO2, ZnO), ketones (e.g. camphor), isethionates, lanolin and derivatives, organic salts, phenols including salts (e.g. parabens), phosphorus compounds (e.g. phosphate derivatives), polyacrylates and acrylate copolymers, protein and enzymes derivatives (e.g. collagen), synthetic polymers including salts, siloxanes and silanes, sorbitan derivatives, sterols, sulfonic acids and derivatives and waxes.

Some examples of antiacne agents are Salicylic acid and Sulfur.

Some examples of antifungal agents are Calcium Undecylenate, Undecylenic Acid, Zinc Undecylenate, and Povidone-Iodine.

Some examples of antimicrobial agents are Alcohol, Benzalkonium Chloride, Benzethonium Chloride, Hydrogen Peroxide, Methylbenzethonium Chloride, Phenol, Poloxamer 188, and Povidone-Iodine.

Some examples of antioxidants are Acetyl Cysteine, Arbutin, Ascorbic Acid, Ascorbic Acid Polypeptide, Ascorbyl Dipalmitate, Ascorbyl Methylsilanol Pectinate, Ascorbyl Palmitate, Ascorbyl Stearate, BHA, p-Hydroxyanisole, BHT, t-Butyl Hydroquinone, Caffeic Acid, Camellia Sinensis Oil, Chitosan Ascorbate, Chitosan Glycolate, Chitosan Salicylate, Chlorogenic Acids, Cysteine, Cysteine HCl, Decyl Mercaptomethylimidazole, Erythorbic Acid, Diamylhydroquinone, Di-t-Butylhydroquinone, Dicetyl Thiodipropionate, Dicyclopentadiene/t-Butylcresol Copolymer, Digalloyl Trioleate, Dilauryl Thiodipropionate, Dimyristyl Thiodipropionate, Dioleyl Tocopheryl Methylsilanol, Isoquercitrin, Diosmine, Disodium Ascorbyl Sulfate, Disodium Rutinyl Disulfate, Distearyl Thiodipropionate, Ditridecyl Thiodipropionate, Dodecyl Gallate, Ethyl Ferulate, Ferulic Acid, Hydroquinone, Hydroxylamine HCl, Hydroxylamine Sulfate, lsooctyl Thioglycolate, Kojic Acid, Madecassicoside, Magnesium Ascorbate, Magnesium Ascorbyl Phosphate, Melatonin, Methoxy-PEG-7 Rutinyl Succinate, Methylene Di-t-Butylcresol, Methylsilanol Ascorbate, Nordihydroguaiaretic Acid, Octyl Gallate, Phenylthioglycolic Acid, Phloroglucinol, Potassium Ascorbyl Tocopheryl Phosphate, Thiodiglycolamide, Potassium Sulfite, Propyl Gallate, Rosmarinic Acid, Rutin, Sodium Ascorbate, Sodium Ascorbyl/Cholesteryl Phosphate, Sodium Bisulfite, Sodium Erythorbate, Sodium Metabisulfide, Sodium Sulfite, Sodium Thioglycolate, Sorbityl Furfural, Tea Tree (Melaleuca Aftemifolia) Oil, Tocopheryl Acetate, Tetrahexyldecyl Ascorbate, Tetrahydrodiferuloylmethane, Tocopheryl Linoleate/Oleate, Thiodiglycol, Tocopheryl Succinate, Thiodiglycolic Acid, Thioglycolic Acid, Thiolacetic Acid, Thiosalicylic Acid, Thiotaurine, Retinol, Tocophereth-5, Tocophereth-10, Tocophereth-12, Tocophereth-18, Tocophereth-50, Tocopherol, Tocophersolan, Tocopheryl Linoleate, Tocopheryl Nicotinate, Tocoquinone, o-Tolyl Biguamide, Tris(Nonylphenyl) Phosphite, Ubiquinone, and Zinc Dibutyldithiocarbamate.

Some examples of cosmetic biocides are Aluminum Phenolsulfonate, Ammonium Phenolsulfonate, Bakuchiol, Benzalkonium Bromide, Benzalkonium Cetyl Phosphate, Benzalkonium Chloride, Benzalkoniumn Saccharinate, Benzethonium Chloride, Potassium Phenoxide, Benzoxiquine, Benzoxonium Chloride, Bispyrithione, Boric Acid, Bromochlorophene, Camphor Benzalkonium Methosulfate, Captan, Cetalkonium Chloride, Cetearalkonium Bromide, Cetethyldimonium Bromide, Cetrimonium Bromide, Cetrimonium Chloride, Cetrimonium Methosulfate, Cetrimonium Saccharinate, Cetrimonium Tosylate, Cetylpyridinium Chloride, Chloramine T, Chlorhexidine, Chlorhexidine Diacetate, Chlorhexidine Digluconate, Chlorhexidine Dihydrochloride, p-Chloro-m-Cresol, Chlorophene, p-Chlorophenol, Chlorothymol, Chloroxylenol, Chlorphenesin, Ciclopirox Olamine, Climbazole, Cloflucarban, Clotrimazole, Coal Tar, Colloidal Sulfur, o-Cymen-5-ol, Dequalinium Acetate, Dequalinium Chloride, Dibromopropamidine Diisethionate, Dichlorobenzyl Alcohol, Dichlorophene, Dichlorophenyl Imidazoldioxolan, Dichloro-m-Xylenol, Diiodomethyltolylsulfone, Dimethylol Ethylene Thiourea, Diphenylmethyl piperazinylbenzimidazole, Domiphen Bromide, 7-Ethylbicyclooxazolidine, Fluorosalan, Formaldehyde, Glutaral, Hexachlorophene, Hexamidine, Hexamidine Diisethionate, Hexamidine Diparaben, Hexamidine Paraben, Hexetidine, Hydrogen Peroxide, Hydroxymethyl Dioxoazabicyclooctane, Ichthammol, Isopropyl Cresol, Lapyrium Chloride, Lauralkonium Bromide, Lauralkonium Chloride, Laurtrimonium Bromide, Laurtrimonium Chloride, Laurtrimonium Trichlorophenoxide, Lauryl Isoquinolinium Bromide, Lauryl Isoquinolinium Saccharinate, Laurylpyridinium Chloride, Mercuric Oxide, Methenamine, Methenammonium Chloride, Methylbenzethonium Chloride, Myristalkonium Chloride, Myristalkonium Saccharinate, Myrtrimonium Bromide, Nonoxynol-9 Iodine, Nonoxynol-12 Iodine, Olealkonium Chloride, Oxyquinoline, Oxyquinoline Benzoate, Oxyquinoline Sulfate, PEG-2 Coco-Benzonium Chloride, PEG-10 Coco-Benzonium Chloride, PEG-6 Undecylenate, PEG-8 Undecylenate, Phenol, o-Phenylphenol, Phenyl Salicylate, Piroctone Olamine, Sulfosuccinylundecylenate, Potassium o-Phenylphenate, Potassium Salicylate, Potassium Troclosene, Propionic Acid, PVP-Iodine, Quaternium-8, Quaternium-14, Quaternium-24, Sodium Phenolsulfonate, Sodium Phenoxide, Sodium o-Phenylphenate, Sodium Shale Oil Sulfonate, Sodium Usnate, Thiabendazole, 2,2′-Thiobis(4-Chlorophenol), Thiram, Triacetin, Triclocarban, Triclosan, Trioctyldodecyl Borate, Undecylenamidopropylamine Oxide, Undecyleneth-6, Undecylenic Acid, Zinc Acetate, Zinc Aspartate, Zinc Borate, Zinc Chloride, Zinc Citrate, Zinc Cysteinate, Zinc Dibutyldithiocarbamate, Zinc Gluconate, Zinc Glutamate, Zinc Lactate, Zinc Phenolsulfonate, Zinc Pyrithione, Zinc Sulfate, and Zinc Undecylenate.

Some examples of external analgesics are Benzyl Alcohol, Capsicum Oleoresin (Capsicum Frutescens Oleoresin), Methyl Salicylate, Camphor, Phenol, Capsaicin, Juniper Tar (Juniperus Oxycedrus Tar), Phenolate Sodium (Sodium Phenoxide), Capsicum (Capsicum Frutescens), Menthol, Resorcinol, Methyl Nicotinate, and Turpentine Oil (Turpentine).

Some examples of oxidizing agents are Ammonium Persulfate, Calcium Peroxide, Hydrogen Peroxide, Magnesium Peroxide, Melamine Peroxide, Potassium Bromate, Potassium Caroate, Potassium Chlorate, Potassium Persulfate, Sodium Bromate, Sodium Carbonate Peroxide, Sodium Chlorate, Sodium Iodate, Sodium Perborate, Sodium Persulfate, Strontium Dioxide, Strontium Peroxide, Urea Peroxide, and Zinc Peroxide.

Some examples of reducing agents are Ammonium Bisufite, Ammonium Sulfite, Ammonium Thioglycolate, Ammonium Thiolactate, Cystemaine HCl, Cystein, Cysteine HCl, Ethanolamine Thioglycolate, Glutathione, Glyceryl Thioglycolate, Glyceryl Thioproprionate, Hydroquinone, p-Hydroxyanisole, Isooctyl Thioglycolate, Magnesium Thioglycolate, Mercaptopropionic Acid, Potassium Metabisulfite, Potassium Sulfite, Potassium Thioglycolate, Sodium Bisulfite, Sodium Hydrosulfite, Sodium Hydroxymethane Sulfonate, Sodium Metabisulfite, Sodium Sulfite, Sodium Thioglycolate, Strontium Thioglycolate, Superoxide Dismutase, Thioglycerin, Thioglycolic Acid, Thiolacetic Acid, Thiosalicylic Acid, and Zinc Formaldehyde Sulfoxylate.

An example of a skin bleaching agent is Hydroquinone.

Some examples of skin protectants are Allantoin, Aluminum Acetate, Aluminum Hydroxide, Aluminum Sulfate, Calamine, Cocoa Butter, Cod Liver Oil, Colloidal Oatmeal, Dimethicone, Glycerin, Kaolin, Lanolin, Mineral Oil, Petrolatum, Shark Liver Oil, Sodium Bicarbonate, Talc, Witch Hazel, Zinc Acetate, Zinc Carbonate, and Zinc Oxide.

Some examples of sunscreen agents are Aminobenzoic Acid, Cinoxate, Diethanolamine Methoxycinnamate, Digalloyl Trioleate, Dioxybenzone, Ethyl 4-[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate.

Some examples of UV light absorbing agents are Acetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, TriPABA Panthenol, Urocanic Acid, and VA/Crotonates/Methacryloxybenzophenone-1 Copolymer.

Typically, mixing all of the components of the composition causes curing to begin at room temperature. As such the component materials can be stored in a plurality of containers prior to use to inhibit curing prior to use. Any of the additional components in the formulation is put in the container that is most desirable.

Typically, the antiperspirant compound of the invention is mixed with the adhesive formulation prior to it curing. Mixing can be performed by any conventional technique. For example, the antiperspirant salts can be added to the adhesive and blended with a mechanical high shear mixing system to disperse the salts. Upon curing or evaporation of carrier solvents, the salts become incorporated into the adhesive matrix.

The mixed composition is then applied to the desired site or, alternatively, the component materials of the invention can be applied onto the desired site in a manner that causes mixing. The composition then cures and forms a cured adhesive. Alternatively, the formulations are applied on a biological surface including, but not limited to animal bodies (e.g., human or other animal) or on substrates which are to be adhered to the human or animal body.

The formulations of the invention can be applied, for example, by coating, rubbing, painting, spraying, or any other conventional method of applying thin films to a substrate such as backing film, release liners, medical device surface. It can also be applied directly to a biological surface such as skin in an in situ curing form.

As noted above, when the formulation is mixed, it cures to form a coating. It may cure, for example, at room temperature or at elevated temperature.

If desired, the adhesive formulation of the invention can be coated on the desired substrate and cured, followed by coating the surface of the adhesive with the antiperspirant compound by, for example, plasma, transfer or spraying coating. If performed in this manner, the antiperspirant can be applied as a pure material, or it can blended in a solvent carrier or be part of a formulation that will form a second layer at the surface of the adhesive.

The final composition can be in the form of a tacky gel (fillerless elastomer), a reinforced elastomer with a tacky surface, a foam or cellular structure or resin.

The formulations and resultant compositions herein are generally acceptable on many biological surfaces such as, for example, skin or other parts of the body such as buccal, oral, nasal, otic or ocular tissues. One of the advantages of the present invention is that it will maintain adherence to most surfaces by reducing the amount of moisture where other adhesives may fail. Specifically, it is postulated that the antiperspirant will inhibit perspiration of the human or other animal body at the site of the adhesive and, thus, allow the adhesive to retain its adhesion.

When used on biological surfaces, the compositions of this invention can be used as, or in, traditional or novel personal hygiene antiperspirant applications. In addition, however, these compositions can be used to adhere devices such as wound dressings, burn treatment, scar management devices, surgical drapes and dressings, medical tapes, ostomies, incontinence devices, electrodes, monitors, chaffing and blister prevention devices, cosmetic or medical prostheses, podiatric devices and other Rx and over-the-counter healthcare and medical device applications where it would be expected that moisture from, for example, perspiration, would decrease adhesion. The invention may also be used to adhere devices that are preventative in nature such as blister prevention, anti-odor devices, joint stabilization devices, and devices that prevent pressure ulcers. The compositions of the invention also find application in recreation and sporting equipment and accessories, including, for example, sports tapes and braces, protective equipment and apparel including falses and masks. Finally, since adhesion to a dry surface typically creates a better seal with the skin, it is expected that the compositions of this invention provide better adhesion than may be achieved with similar adhesives that do not contain antiperspirants.

The composition may also be formed on a film that is then placed on the skin such as a bandage The adhesive film can be formed on the substrate to be adhered or it may be formed on intact or damaged skin.

The resultant films are typically thin and tacky. Films on the order of up to 20 mils (e.g., 0.1 to 15 mils) are often obtained. These films can have many physical properties from gels to elastomers to resins.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %.

The compositions were prepared by mixing the antiperspirant salts at the concentrations listed in Table 1 in the adhesives listed in Table 1. All samples were mixed as simple blends using a Hamilton Beach malt mixer with a Variac speed controller.

In the case of Dow Corning® 7-9800 samples, a 1:1 ratio of 7-9800 A:7-9800 B was mixed together and the antiperspirant salts at the appropriate amount were blended into the mixture. The mixture was then cast and oven cured to form a film of approximately 8 mil thickness.

In the case of Dow Corning® BIO-PSA 7-4502 (60% solids content in ethyl acetate carrier solvent) samples, the solvated adhesive was mixed with the appropriate amount of antiperspirant salts to provide the 5% or 30% salt concentration after the solvent was driven off. The solution was cast at approximately 30 mil thickness.

TABLE 1

| Antiperspirant | Concentration | Adhesive |
|---|---|---|
| REACH 908 SUF* | 30% | 7-9800** Soft Skin Adhesive |
| REACH 908 SUF* | 5% | 7-9800** Soft Skin Adhesive |
| REACH 908 SUF* | 30% | 7-4502*** after devolatizing |
| | | the solvent |
| REACH 908 SUF* | 5% | 7-4502*** after devolatizing |
| | | the solvent |

*REACH 908 SUF is an aluminum-zirconium tetrachlorohydrex GLY salt sold by Reheis, Inc.
**7-9800 is the reaction product of a dimethylvinylsiloxy terminated dimethyl siloxane, trimethylsiloxy terminated dimethyl, methyl hydrogen siloxane and a hydrogen terminated dimethyl siloxane in the presence of a platinum catalyst.
***7-4502 is the reaction product of hydroxyl terminated dimethylsiloxane and a silicone resin diluted to 60 wt. % solids in a solvent

The 7-9800 showed good incorporation of the salts into the 7-9800 matrix. Adhesion and cohesion, although still reasonable, were slightly less than similarly prepared samples without the antiperspirant salts as determined by subjective touching.

The 7-4502 samples also had slightly diminished tack over controls without the antiperspirant salts, but cohesion was still very strong. Both were also determined by subjective methods.

That which is claimed is:

1. A composition comprising:
1 to 99.9 wt. % of a silicone-containing adhesive formulation; and
0.1 to 50 wt. % antiperspirant compound.

2. The composition according to claim 1 wherein the formulation also comprises hygroscopic filler or active agents.

3. The composition of claim 1 wherein silicone-containing adhesive formulation comprises at least one polydiorganosiloxane having silicon-bonded alkenyl groups, at least one hydrosilicon compound having silicon bonded hydrogen atoms and a catalyst for the reaction of the Si—H groups with the Si-alkenyl groups.

4. The composition of claim 3 also comprising a hydroxy-substituted siloxane resin.

5. The composition of claim 1 wherein silicone-containing adhesive formulation comprises the condensation reaction product of a polydiorganosiloxne having silanol terminal functionality and a silanol containing silicone resin.

6. The composition of claim 1 wherein silicone-containing adhesive formulation comprises a hot-melt silicone pressure sensitive adhesive.

7. The composition of claim 1 wherein the antiperspirant is selected from the group consisting of aluminum containing salts, zirconium containing salts and mixtures thereof.

8. A method of enhancing the adhesion of a silicone containing adhesive formulation comprising:
mixing 1 to 99.9 wt. % of a silicone-containing adhesive formulation and 0.1 to 50 wt. % antiperspirant compound to form a composition; and
applying the composition onto a surface and allowing it to cure.

9. A method of adhering a medical device to skin comprising:
mixing a composition comprising 1 to 99.9 wt. % of a silicone-containing adhesive formulation and 0.1 to 50 wt. % of an antiperspirant compound to form a composition;
applying the composition onto a surface of a medical device; and
adhering the medical device to the skin by placing a surface of the medical device containing the composition onto the skin.

10. A method of adhering a medical device to skin comprising:
mixing a composition comprising 1 to 99.9 wt. % of a silicone-containing adhesive formulation and 0.1 to 50 wt. % of an antiperspirant compound to form a composition;
applying the composition onto skin; and
placing a medical device onto the skin having the composition thereon.

11. The method according to claim 8 wherein the surface comprises human skin.

12. A method of enhancing the adhesion of a silicone containing adhesive formulation comprising:
applying a silicone-containing adhesive formulation onto a substrate and allowing it to cure; and
applying an antiperspirant compound onto a surface of the cured adhesive formulation.

13. The method of claim 12 wherein the substrate is skin and wherein a medical device is placed on the cured adhesive formulation having the antiperspirant compound applied thereon.

14. The method of claim 12 wherein the substrate is a medical device and wherein a surface of the medical device having the cured adhesive formulation and the antiperspirant compound applied thereon onto the skin.
